(19) United States
(12) Patent Application Publication
(10) Pub. No.: US 2021/0052159 A1
(43) Pub. Date: Feb. 25, 2021

(54) TECHNIQUES FOR PROVIDING HEALTH ASSISTANCE INFORMATION

(71) Applicant: QUALCOMM Incorporated, San Diego, CA (US)

(72) Inventor: Arnold Jason GUM, San Diego, CA (US)

(21) Appl. No.: 16/548,636

(22) Filed: Aug. 22, 2019

(51) Int. Cl. A61B 5/00; H04W 4/021; G08B 21/02; G08B 31/00; G16H 50/20; A61B 5/11; A61B 5/0205; A61B 5/145; A61B 5/1455; A61B 5/02; A61B 5/046; A61B 5/0464

(52) U.S. Cl. CPC A61B 5/0022; H04W 4/021; G08B 21/02; G08B 31/00; G16H 50/20; A61B 5/747; H04M 1/72538; A61B 5/02055; A61B 5/14532; A61B 5/14551; A61B 5/02028; A61B 5/046; A61B 5/0464; A61B 5/1112

(57) ABSTRACT

Certain aspects of the present disclosure generally relate to providing health assistance information. In some aspects, a device determines one or more rates of change for one or more physiological parameters of a user, and may determine whether the one or more rates of change meets one or more thresholds. The device, in response to the determination that the one or more rates of change meets one or more thresholds, may identify one or more potential user conditions based on the one or more rates of change, a location of the device and user profile. The device may provide one or more messages based on the one or more potential user conditions.

### FIELD OF THE DISCLOSURE

Aspects of the present disclosure generally relate to techniques for providing health assistance information.

### BACKGROUND

Various changes within a user's body may be indicative of an underlying issue or problem, but the user may be unaware of these various changes until the underlying issue or problem has progressed to a later stage with more noticeable symptoms. By addressing these issues early on, a user may be able to reduce the symptoms that are more burdensome to the user due to the underlying issue. Also, sometimes underlying issues are associated with user behavior, eating, or drinking patterns; however, associating particular behavior and eating patterns with a specific condition may be challenging, especially where symptoms are not acute and/or immediate.

### SUMMARY

Certain aspects of the present disclosure generally relate to providing health assistance information. In some aspects, a device may include one or more sensors, one or more memory and one or more processors, wherein the one or more processors are communicatively coupled to the one or more memory and the one or more sensors and the one or more processors are configured to determine one or more rates of change for one or more physiological parameters of a user, and determine whether the one or more rates of change meets one or more thresholds. The one or more processors, in response to the determination that the one or more rates of change meets one or more thresholds, are configured to identify one or more potential user conditions based on the one or more rates of change, a location of the device and user profile and provide one or more messages based on the one or more potential user conditions.

In some aspects, a method may include determining, at a first device, one or more rates of change for one or more physiological parameters of a user, and determining, at the first device, whether the one or more rates of change meets one or more thresholds. The method may include in response to the determination that the one or more rates of change meets one or more thresholds, identifying, at the first device, one or more potential user conditions based on the one or more rates of change, a location of the device and user profile. The method may include providing one or more messages based on the one or more potential user conditions.

In some aspects, a device comprises means for determining one or more rates of change for one or more physiological parameters of a user, and means for determining whether the one or more rates of change meets one or more thresholds. The device, in response to the determination that the one or more rates of change meets one or more thresholds, may include means for identifying one or more potential user conditions based on the one or more rates of change, a location of the device and user profile. The device may include means for providing one or more messages based on the one or more potential user conditions.

In some aspects, a non-transitory computer-readable medium comprises processor-executable code configured to cause one or more processors to determine one or more rates of change for one or more physiological parameters of a user, and may determine whether the one or more rates of change meets one or more thresholds. The non-transitory computer-readable medium, in response to the determination that the one or more rates of change meets one or more thresholds, may identify one or more potential user conditions based on the one or more rates of change, a location of the device and user profile. The non-transitory computer-readable medium may provide one or more messages based on the one or more potential user conditions.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects. The same reference numbers in different drawings may identify the same or similar elements.

FIG. 1 shows an example of a communication environment in which various aspects of the disclosure may be implemented.

FIG. 2 shows an example process diagram illustrating a method of determining one or more user conditions.

FIG. 3 shows an example process diagram illustrating a method of associating one or more rates of change with a venue.

FIG. 4 shows an example process diagram illustrating a method of identifying a consumed product based on changes to a user's physiological parameter(s).

FIG. 5 shows an example process diagram illustrating a method of deriving a user-specific profile from a user-similar profile.

FIG. 6 shows an example process diagram illustrating a method of associating physiological parameters and user conditions

FIG. 7 shows an example process diagram illustrating a method of determining a user condition.

FIG. 8 shows an example table for illustrating a user profile.

FIG. 9 shows an example table for illustrative how various responses are associated with different parameters.

FIG. 10 shows an example table for a consumption profile.

FIG. 11 is an example device and the components within the device in which aspects of the disclosure may be implemented.

FIG. 12 is an example server and the components within the server in which aspects of the disclosure may be implemented.

### DETAILED DESCRIPTION

The detailed description set forth below, in connection with the appended drawings, is intended as a description of various configurations and is not intended to represent the only configurations in which the concepts described herein may be practiced. The detailed description includes specific details for providing a thorough understanding of the various concepts. However, it will be apparent to those skilled in the art that these concepts may be practiced without these specific details.

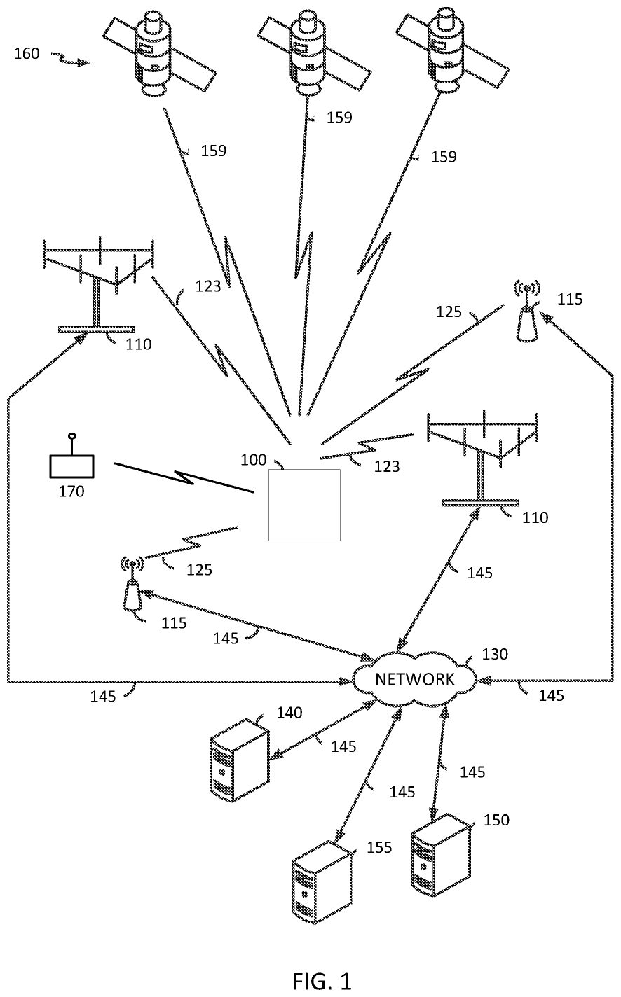

As shown in FIG. 1 in a particular implementation, mobile device 100, which may also be referred to as a UE (or user equipment), may transmit radio signals to, and receive radio signals from, a wireless communication network. In one example, mobile device 100 may communicate with a cellular communication network by transmitting wireless signals to, or receiving wireless signals from a cellular transceiver 110 which may comprise a wireless base transceiver subsystem (BTS), a Node B or an evolved NodeB (eNodeB) over wireless communication link 123. Similarly, mobile device 100 may transmit wireless signals to, or receive wireless signals from local transceiver 115 over wireless communication link 125. A local transceiver 115 may comprise an access point (AP), femtocell, Home Base Station, small cell base station, Home Node B (HNB) or Home eNodeB (HeNB) and may provide access to a wireless local area network (WLAN, e.g., IEEE 802.11 network), a wireless personal area network (WPAN, e.g., Bluetooth® network) or a cellular network (e.g. an LTE network or other wireless wide area network such as those discussed in the next paragraph). Of course it should be understood that these are merely examples of networks that may communicate with a mobile device over a wireless link, and claimed subject matter is not limited in this respect.

Examples of network technologies that may support wireless communication link 123 are Global System for Mobile Communications (GSM), Code Division Multiple Access (CDMA), Wideband CDMA (WCDMA), Long Term Evolution LTE), High Rate Packet Data (HRPD). GSM, WCDMA, LTE, 5G NR are technologies defined by 3GPP. CDMA and HRPD are technologies defined by the 3^{rd} Generation Partnership Project 2 (3GPP2). WCDMA is also part of the Universal Mobile Telecommunications System (UMTS) and may be supported by an HNB. Cellular transceivers 110 may comprise deployments of equipment providing subscriber access to a wireless telecommunication network for a service (e.g., under a service contract). Here, a cellular transceiver 110 may perform functions of a cellular base station in servicing subscriber devices within a cell determined based, at least in part, on a range at which the cellular transceiver 110 is capable of providing access service. Examples of radio technologies that may support wireless communication link 125 are IEEE 802.11, Bluetooth (BT) and LTE.

In a particular implementation, cellular transceiver 110 and local transceiver 115 may communicate with servers 140, 150 and/or 155 over a network 130 through links 145. Here, network 130 may comprise any combination of wired or wireless links and may include cellular transceiver 110 and/or local transceiver 115 and/or servers 140, 150 and 155. In a particular implementation, network 130 may comprise Internet Protocol (IP) or other infrastructure capable of facilitating communication between mobile device 100 and servers 140, 150 or 155 through local transceiver 115 or cellular transceiver 110. In an embodiment, network 130 may also facilitate communication between mobile device 100, servers 140, 150 and/or 155 and a public safety answering point (PSAP) 160, for example through communications link 165) In another implementation, network 130 may comprise cellular communication network infrastructure such as, for example, a base station controller or packet based or circuit based switching center (not shown) to facilitate mobile cellular communication with mobile device 100. In a particular implementation, network 130 may comprise local area network (LAN) elements such as WiFi APs, routers and bridges and may in that case include or have links to gateway elements that provide access to wide area networks such as the Internet. In other implementations, network 130 may comprise a LAN and may or may not have access to a wide area network but may not provide any such access (if supported) to mobile device 100. In some implementations network 130 may comprise multiple networks (e.g., one or more wireless networks and/or the Internet). In one implementation, network 130 may include one or more serving gateways or Packet Data Network gateways. In addition, one or more of servers 140, 150 and 155 may be an E-SMLC, a Secure User Plane Location (SUPL) Location Platform (SLP), a SUPL Location Center (SLC), a SUPL Positioning Center (SPC), a Position Determining Entity (PDE) and/or a gateway mobile location center (GMLC), each of which may connect to one or more location retrieval functions (LRFs) and/or mobility management entities (MMEs) in network 130. The servers 140, 150 and/or 155 may be location server, point of interest server, navigation routing server, health server, crowdsource server, application server, etc.

In particular implementations, and as discussed below, mobile device 100 may have circuitry and processing resources capable of obtaining location related measurements (e.g. for signals received from GPS or other Satellite Positioning System (SPS) satellites 160, cellular transceiver 110 or local transceiver 115 and possibly computing a position fix or estimated location of mobile device 100 based on these location related measurements. In some implementations, location related measurements obtained by mobile device 100 may be transferred to a location server such as an enhanced serving mobile location center (E-SMLC) or SUPL location platform (SLP) (e.g. which may be one of servers 140, 150 and 155) after which the location server may estimate or determine a location for mobile device 100 based on the measurements. In the presently illustrated example, location related measurements obtained by mobile device 100 may include measurements of signals (159) received from satellites belonging to an SPS or Global Navigation Satellite System (GNSS) such as GPS, GLONASS, Galileo or Beidou 160 and/or may include measurements of signals (such as 123 and/or 125) received from terrestrial transmitters fixed at known locations (e.g., such as cellular transceiver 110). Mobile device 100 or a separate location server may then obtain a location estimate for mobile device 100 based on these location related measurements using any one of several position methods such as, for example, GNSS, Assisted GNSS (A-GNSS), Advanced Forward Link Trilateration (AFLT), Observed Time Difference Of Arrival (OTDOA) or Enhanced Cell ID (E-CID) or combinations thereof. In some of these techniques (e.g. A-GNSS, AFLT and OTDOA), pseudoranges or timing differences may be measured at mobile device 100 relative to three or more terrestrial transmitters fixed at known locations or relative to four or more satellites with accurately known orbital data, or combinations thereof, based at least in part, on pilots, positioning reference signals (PRS) or other positioning related signals transmitted by the transmitters or satellites and received at mobile device 100. Here, servers 140, 150 or 155 may be capable of providing positioning assistance data to mobile device 100 including, for example, information regarding signals to be measured (e.g., signal timing), locations and identities of terrestrial transmitters and/or signal, timing and orbital information for GNSS satellites to facilitate positioning techniques such as A-GNSS, AFLT, OTDOA and E-CID. For example, servers 140, 150 or 155 may comprise an almanac which indicates locations and identities of cellular transceivers and/or local transceivers in a particular region or regions such as a particular venue, and may provide information descriptive of signals transmitted by a cellular base station or AP such as transmission power and signal timing. In the case of E-CID, a mobile device 100 may obtain measurements of signal strengths for signals received from cellular transceiver 110 and/or local transceiver 115 and/or may obtain a round trip signal propagation time (RTT) between mobile device 100 and a cellular transceiver 110 or local transceiver 115. A mobile device 100 may use these measurements together with assistance data (e.g. terrestrial almanac data or GNSS satellite data such as GNSS Almanac and/or GNSS Ephemeris information) received from a server 140, 150 or 155 to determine a location for mobile device 100 or may transfer the measurements to a server 140, 150 or 155 to perform the same determination. A call from mobile device 100 may be routed, based on the location of mobile device 100, and connected to a Public Safety Answering Point (PSAP) 160, for example, via wireless communication link 123 and communications link 165. PSAP may, in an embodiment, correspond to PSAP or a legacy PSAP. As noted earlier, the servers 140, 150 and/or 155 may also be health server that provide health information (e.g. how physiological parameters may change to indicate one or more user conditions and/or symptoms) to the wearable device 170 and/or mobile device 100, it may be used to crowdsource health information, it may be an insurance server that obtains information (e.g. movement, medication compliance, etc) from the mobile device 100 and/or wearable device 170, etc.

A mobile device (e.g. mobile device 100 in FIG. 1) may be referred to as a device, a wireless device, a mobile terminal, a terminal, a mobile station (MS), a user equipment (UE), a SUPL Enabled Terminal (SET) or by some other name and may correspond to a cellphone, smartphone, laptop, tablet, PDA, tracking device, wearable device (e.g. smart watch, diagnostic device, clothing, etc), medical diagnostic device, interest of things/everything device (IOT) or some other portable or moveable device. Typically, though not necessarily, a mobile device may support wireless communication such as using GSM, WCDMA, LTE, CDMA, HRPD, WiFi, BT, WiMax, etc. A mobile device may also support wireless communication using a wireless LAN (WLAN), DSL or packet cable for example. A mobile device may comprise a single entity or may comprise multiple entities such as in a personal area network where a user may employ audio, video and/or data I/O devices and/or body sensors and a separate wireline or wireless modem. An estimate of a location of a mobile device (e.g., mobile device 100) may be referred to as a location, location estimate, location fix, fix, position, position estimate or position fix, and may be geographic, thus providing location coordinates for the mobile device (e.g., latitude and longitude) which may or may not include an altitude component (e.g., height above sea level, height above or depth below ground level, floor level or basement level). Alternatively, a location of a mobile device may be expressed as a civic location (e.g., as a postal address or the designation of some point or small area in a building such as a particular room or floor). A location of a mobile device may also be expressed as an area or volume (defined either geographically or in civic form) within which the mobile device is expected to be located with some probability or confidence level (e.g., 67% or 95%). A location of a mobile device may further be a relative location comprising, for example, a distance and direction or relative X, Y (and Z) coordinates defined relative to some origin at a known location which may be defined geographically or in civic terms or by reference to a point, area or volume indicated on a map, floor plan or building plan. In the description contained herein, the use of the term location may comprise any of these variants unless indicated otherwise.

The mobile device 100 may also communicate with a wearable device 170. For example, the mobile device 100 may be a smartphone and the wearable device 170 may be a smart watch with one or more sensors. As described throughout the specification, a mobile device may be mobile 100 or a wearable device 170. The wearable device 170 or mobile device 100 may have one or more sensors, such as a photoplethysmography (PPG), electrocardiography (ECG), peripheral capillary oxygen saturation (SpO2), blood pressure and/or pulse transit time sensor, body temperature sensor, ultrasonic sensor/acoustic sensor, pressure sensor, heart rate sensor, respiration rate sensor, electrodermal activity (EDA), inertial motion unit sensors (IMU), photoacoustic sensors, blood alcohol, pressure cuff, moisture sensor, chemical sensors, gas sensors, conductivity sensor, microphone(s), speaker(s), etc. The wearable device 170 may also include any component listed in mobile device 100, such as GNSS receiver, Display, etc.

The mobile device 100 may communication with a wearable device 170 over a personal area network, such as Bluetooth® Low Energy, a local area network, such as WLAN, and/or over a WWAN, such as 5G. The wearable device 170 may communicate with a network directly via a PAN, WLAN and/or WWAN, and/or may communicate through a mobile device 100.

The wearable device 170 may be a smart watch, head mount display (such as virtual reality or augmented reality headset), smart clothing (e.g. shirts, pants, gloves, etc) or footwear (e.g. sock, shoes, sandals, etc), fitness band, health diagnostics device, jewelry (e.g. rings, necklaces), a patch (e.g. may be adhered to a person's skin), etc.

Reference throughout this specification to “one example”, “an example”, “certain examples”, or “exemplary implementation” means that a particular feature, structure, or characteristic described in connection with the feature and/or example may be included in at least one feature and/or example of claimed subject matter. Thus, the appearances of the phrase “in one example”, “an example”, “in certain examples” or “in certain implementations” or other like phrases in various places throughout this specification are not necessarily all referring to the same feature, example, and/or limitation. Furthermore, the particular features, structures, or characteristics may be combined in one or more examples and/or features.

Some portions of the detailed description included herein are presented in terms of algorithms or symbolic representations of operations on binary digital signals stored within a memory of a specific apparatus or special purpose computing device or platform. In the context of this particular specification, the term specific apparatus or the like includes a general-purpose computer once it is programmed to perform particular operations pursuant to instructions from program software. Algorithmic descriptions or symbolic representations are examples of techniques used by those of ordinary skill in the signal processing or related arts to convey the substance of their work to others skilled in the art. An algorithm is here, and generally, is considered to be a self-consistent sequence of operations or similar signal processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals, or the like. It should be understood, however, that all of these or similar terms are to be associated with appropriate physical quantities and are merely convenient labels. Unless specifically stated otherwise, as apparent from the discussion herein, it is appreciated that throughout this specification discussions utilizing terms such as “processing,” “computing,” “calculating,” “determining” or the like refer to actions or processes of a specific apparatus, such as a special purpose computer, special purpose computing apparatus or a similar special purpose electronic computing device. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device.

Wireless communication techniques described herein may be in connection with various wireless communications networks such as a wireless wide area network (“WWAN”), a wireless local area network (“WLAN”), a wireless personal area network (WPAN), and so on. The term “network” and “system” may be used interchangeably herein. A WWAN may be a Code Division Multiple Access (“CDMA”) network, a Time Division Multiple Access (“TDMA”) network, a Frequency Division Multiple Access (“FDMA”) network, an Orthogonal Frequency Division Multiple Access (“OFDMA”) network, a Single-Carrier Frequency Division Multiple Access (“SC-FDMA”) network, or any combination of the above networks, and so on. A CDMA network may implement one or more radio access technologies (“RATs”) such as cdma2000, Wideband-CDMA (“W-CDMA”), to name just a few radio technologies. Here, cdma2000 may include technologies implemented according to IS-95, IS-2000, and IS-856 standards. A TDMA network may implement Global System for Mobile Communications (“GSM”), Digital Advanced Mobile Phone System (“D-AMPS”), or some other RAT. GSM and W-CDMA are described in documents from a consortium named “3rd Generation Partnership Project” (“3GPP”). Cdma2000 is described in documents from a consortium named “3rd Generation Partnership Project 2” (“3GPP2”). 3GPP and 3GPP2 documents are publicly available. 4G Long Term Evolution (“LTE”) communications networks may also be implemented in accordance with claimed subject matter, in an aspect. A WLAN may comprise an IEEE 802.11x network, and a WPAN may comprise a Bluetooth network, an IEEE 802.15x, for example. Wireless communication implementations described herein may also be used in connection with any combination of WWAN, WLAN or WPAN.

While there has been illustrated and described what are presently considered to be example features, it will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein.

Therefore, it is intended that claimed subject matter is not limited to the particular examples disclosed, but that such claimed subject matter may also include all aspects falling within the scope of appended claims, and equivalents thereof.

For an implementation involving firmware and/or software, the methodologies may be implemented with modules (e.g., procedures, functions, and so on) that perform the functions described herein. Any machine-readable medium tangibly embodying instructions may be used in implementing the methodologies described herein. For example, software codes may be stored in a memory and executed by a processor unit. Memory may be implemented within the processor unit or external to the processor unit. As used herein the term “memory” refers to any type of long term, short term, volatile, nonvolatile, or other memory and is not to be limited to any particular type of memory or number of memories, or type of media upon which memory is stored.

If implemented in firmware and/or software, the functions may be stored as one or more instructions or code on a computer-readable storage medium. Examples include computer-readable media encoded with a data structure and computer-readable media encoded with a computer program. Computer-readable media includes physical computer storage media. A storage medium may be any available medium that can be accessed by a computer. By way of example, and not limitation, such computer-readable media can comprise RAM, ROM, EEPROM, CD-ROM or other optical disk storage, magnetic disk storage, semiconductor storage, or other storage devices, or any other medium that can be used to store desired program code in the form of instructions or data structures and that can be accessed by a computer; disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media.

In addition to storage on computer-readable storage medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims. That is, the communication apparatus includes transmission media with signals indicative of information to perform disclosed functions. At a first time, the transmission media included in the communication apparatus may include a first portion of the information to perform the disclosed functions, while at a second time the transmission media included in the communication apparatus may include a second portion of the information to perform the disclosed functions.

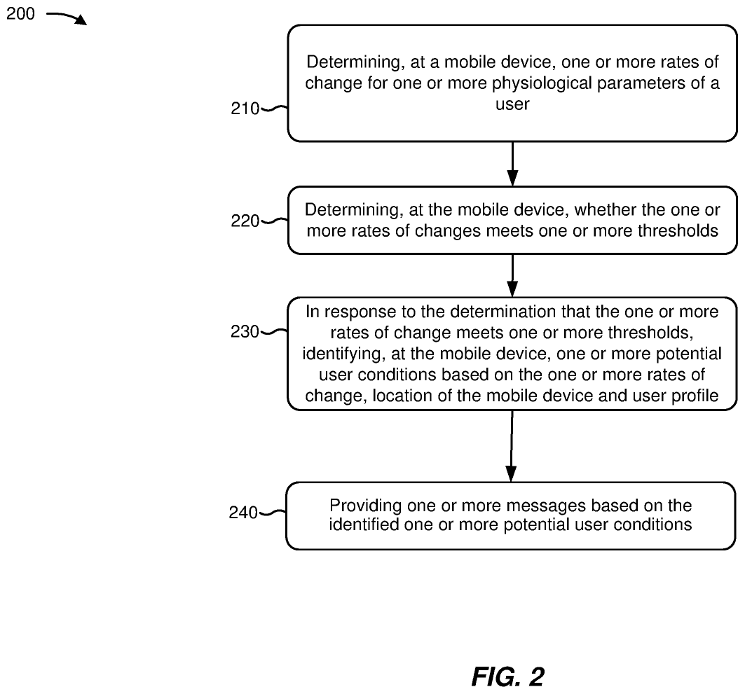

FIG. 2 is a process diagram 200 illustrating an example method of determining one or more potential user conditions. While this process is illustrated with a mobile device 100 and/or a wearable device 170, a server 140 may also be used. The measurements performed may be done on a wearable device 170 and/or on a mobile device 100, but the measurements may be provided to a mobile device 100 and/or a server 140 for the process diagram 200 to proceed.

At block 210, a mobile device 100 and/or a wearable device 170 determines one or more rates of change for one or more physiological parameters of a user. The one or more physiological parameters of a user may comprise blood pressure, pule transit time, heart rate, glucose levels, respiratory rate, peripheral capillary oxygen saturation, stroke volume, cardiac output, heart rhythm, body temperature, skin temperature, triglycerides, skin conductivity/sweat sensor, muscle contraction or any combination thereof.

While rate of change is used throughout the specification, it is important to note this may be a total amount of change over a particular time period in combination with rate of change or alternatively to rate of change. For example, it may be twenty bpm increase over an hour.

A mobile device 100 and/or a wearable 170 determine or indicate the one or more rates of change in absolute values, relative values, percentages, etc. For example, if there is a 20 millimeters of mercury (mmHg) spike in blood pressure for both systolic and diastolic, it may be reported as such, but if it is reported in absolute terms and the baseline was 120 systolic and diastolic being 80 then in absolute terms the new measurement would be 140 systolic over 100 diastolic. If reported by percentage it would be ˜16% increase for systolic and 25% increase for diastolic. This may be indicated based on the last measurement performed by the device, it may indicate over what time period this has occurred (e.g. five minute window, over the last thirty minutes, etc), or this may be standardized information (e.g. OEM specified, industry standard body defined, etc).

If there are a plurality of physiological parameters, the physiological parameters may be determined and/or obtained simultaneously and/or may be obtained at different time periods (e.g. sequentially, partially sequential, or partially different time periods). The physiological parameters may be obtained periodically, quasi-periodically and/or on-demand. The term “quasi-periodically” refers to an event that occurs periodically with a frequency that may change from time to time, and/or to an event occurs from time to time with no well-defined frequency. On-demand means when an application or a user requests a measurement take place immediately or at a particular time.

In one embodiment, the one or more rates of change, total change over a period of time and/or absolute values of one or more physiological parameters may be transmitted to a crowdsourcing server and may be anonymized. The crowdsourcing server may be used to identify various user conditions and/or symptoms over a large group of people and/or identify side effects from medication and/or probabilities of side effects from medication.

The one or more rates of change, total change over a period of time and/or absolute values of one or more physiological parameters may also be transmitted to a health server for the health server to identify one or more user conditions and/or symptoms. For example, the user may be unaware of a particular user condition and/or symptoms, but the health server can use crowdsourced data, clinical data, etc. to detect and determine the user condition(s) and/or symptom(s).

At block 220, the mobile device 100 and/or the wearable device 170 may determine whether the one or more rates of change meets one or more thresholds. The one or more thresholds may be OEM defined, service operator defined, insurance operator defined, user-specific, user-similar or any combination thereof. Service operator defined thresholds are thresholds that are provided by the service operator of the device. Insurance operator defined may be the insurance provider for the user or the one that provided the device, such as United Healthcare®. It is important to note there may more than one threshold and there may be a one or more thresholds that may all be met before a device determines the one or more rates of change should be used to identify one or more user conditions and/or symptoms. For example, a device may have a first threshold that indicates a twenty beats per minute increase over a five minute window, but may also determine that there has been a forty beats per minute increase over the last sixty minutes. In this scenario, even though the rates of change are for the same physiological parameter there are two thresholds that should be met for the device to proceed to block 230. The device may also have thresholds for different physiological parameters that should be met before it proceeds. The thresholds may be called thresholds, sub-thresholds, micro-thresholds, etc.

The device may also have one or more thresholds for the one or more rates of change, but may also have one or more thresholds for the physiological parameter value. For example, it may need a twenty beats per minute increase over a five-minute window but that the device also determines a heart rate of over one hundred and sixty bpm prior to determining a user's condition and/or symptoms.

The user-similar thresholds may be thresholds that are from one or more other users that have one or more similarities to the user. These similarities may comprise use build/shape, weight, water weight, muscle weight, bone mass, heart rate, blood pressure, height, ethnicity, age, gender, or any combination thereof.

The thresholds may be based on a user-specific baseline, historical patterns of the one or more physiological parameters or any combination thereof.

The user-specific baseline may be determined over a period of time. There may be a baseline that is determined for the user for when the user is resting/sleeping, active but the one or more physiological parameters have little to no variation, etc. The baseline may be used to establish the threshold. It may have preestablished thresholds to use based on the baseline (e.g. the threshold may always be 25 mmHg from baseline).

According to an aspect of the disclosure, the threshold may be determined for the user. For example, if over a period of exercising, the user's variability for heart rate is only ten beats per minute (bpm), then the threshold may be set to ten bpm. The threshold may also include a buffer so if the variability for heart rate is always ten bpm, then the threshold may be set to 12 bpm.

The buffer for a threshold may be predefined or dynamically determined. It may be based on a statistic related to one or more physiological parameters. For example, if variability of the heart rate is used to establish a threshold, then instead of using the variability value as the threshold the device may determine a statistic associated with the variability value and use that in combination with the variability value. The statistics may be a percentage, standard deviation, average, mean, etc. For example, if the variability of a user's heart rate is ten bpm, and the statistics used is twenty percent of the variability then the threshold for heart rate may be set to twelve bpm.

Historical patterns of the one or more physiological parameters may be used to establish a threshold. For example, if after a user exercises there is a sharp spike is blood pressure

The thresholds may be absolute thresholds or relative thresholds. For example, the absolute threshold may be a user heart rate of one hundred and sixty beats per minute whereas a relative threshold may be a user heart rate of two beats per minute increase in a five-minute window. The thresholds may be activity specific, such that different activities have different baseline thresholds or generic such that a given threshold is used across multiple activities.

At block 230, the mobile device 100 and/or wearable device 170 may in response to the determination that the one or more rates of changes meets one or more thresholds, identify one or more potential user conditions based on the one or more rates of change, a location of the mobile device and user profile.

The one or more potential user conditions may comprise diabetic reaction, choking, asthma attack, allergic reaction, hypertension, hypoglycemia, atrial fibrillation, tachycardia, a medication controlled medical condition, or any combination thereof.

The mobile device 100 and/or wearable 170 may identify one or more potential user conditions by comparing the one or more rates of change to expected rates of change associated with one or more user conditions from a plurality of user conditions. The user conditions that have one or more rates of change with similarities to the current one or more rates of change are identified and those are provided as potential user conditions.

In one embodiment, the device may provide the one or more potential user conditions to a user via a display (and/or speaker) and the device may receive a user's selection of one or more of the potential user condition(s).

According to an aspect of the disclosure, the device may transmit the one or more potential user conditions to a server 140 for further validation, information, etc. For example, the device may provide the one or more rates of change (or one of the other parameters discussed in the specification), and this may be alternatively or in addition to the user conditions and/or symptoms. The server 140 may request additional information (e.g. new physiological parameters, trends over a period of time, request information from a third party, etc.). For example, it may request validation by a human, such as a doctor, and the doctor may specifically identify the user condition and provide it to the server, and the server may provide that information (and/or treatment options, medication, etc.) to the device.

The mobile device 100 and/or wearable 170 may determine a location of the device. One or more venues are determined based on the location. For example, there may be a point of interest database that indicates venues and locations of those venues, so the location is compared to locations in the database and those locations are used to return one or more venues.

The venue may be used to determine whether to identify the potential user conditions or whether to provide the identified user conditions. If the venue is associated to a place where the rate of change of one or more physiological parameters are expected vary then the mobile device 100 and/or wearable device 170 may ignore the identified user conditions or may not identify the potential user conditions.

According to an aspect of the disclosure, the venue may be associated with ranges for the rates of changes for one or more physiological parameters. If the rate of change for one or more physiological parameter for a user meets or exceeds (at the upper bound) or meets or falls below (at the lower bound) the range(s) then the device may provide one or more messages based on the identified user conditions or the device may proceed with identifying one or more user conditions.

The venue may be associated with one or more thresholds. If the rate of change meets or exceeds the threshold(s) or meets and falls below these threshold(s) (if it is a lower bound) then the mobile device 100 and/or wearable device 170 may proceed with identifying the potential one or more user conditions or providing the identified one or more potential user conditions.

The device may determine whether one or more rates of change correspond to one or more conditions related to the venue. For example, if the venue is a gym then venue thresholds may be used to determine whether the one or more rates of changes are within the thresholds for the venue. If it is meets or exceeds the thresholds or the device determines the one or more rates of changes are not related to the one or more conditions related to the venue, the device may determine one or more potential user conditions based on the one or more rates of change and the user profile.

The device may determine whether one or more rates of changes correspond to one or more activities. For example, the device may identify or detect motion patterns that indicate a particular activity, such as running, walking on stairs, rowing, weight lifting, etc. This may be combined with venue information to provide further information, such as instead of just running the activity to be further narrowed to running on a treadmill (e.g. venue is a gym).

In one embodiment, the device may obtain information from a second device, wherein the second device provides information for the device to determine the activity. The second device may indicate potential activities in the venue, such as treadmill, elliptical, weight lifting, etc. According to an aspect of the disclosure, the second device may indicate the device type (e.g. treadmill, refrigerator, stove, health diagnostic device).

According to an aspect of the disclosure, the device may have a near field communication (NFC) reader (or similar) that reads an NFC tag (or another RF tag) that is attached to a consumable product (e.g. medication, beverage, food). The device may use this information to identify the consumable product and determine how the user's physiological parameters will respond to consuming the product. It may then be able to provide information to the user prior to them consuming the product (e.g. “Don't do that, because you'll be up all night if you drink that coffee”). This may be similarly be done with other technology such as computer vision.

In one embodiment, the device may have communication capabilities (e.g. 5G NR) that allows it to communicate with a second device (e.g. IOT device, refrigerator, etc). The second device may have various capabilities, such as camera/computer vision, NFC reader, etc. The second device may determine what a user that is associated with the device is reaching for and/or about to consume. For example, the second device may be a refrigerator and may determine that the user is trying to reach for a soda (e.g. via computer vision and/or NFC tags), so the refrigerator may send a message to the user device and the user device may provide a notification to the user to avoid drinking the soda.

The user profile may include user conditions to which the user may have or be susceptible. For example, it may indicate that the user has asthma and takes blood pressure medication. This may be used to determine potential user condition or may weight particular user conditions more heavily based on the user profile information. For example, if the respiratory rate drops, then a preliminary potential user condition list may be choking or asthma attack, but if the user profile indicates that the user has asthma then the potential user condition may be set to asthma attack.

The user profile may also contain the one or more thresholds. These thresholds may be used in block 210, may be user specific, may be user specific when in a venue, may be user similar when in a venue, may be venue specific (but not specific to the user is any way), etc.

FIG. 8 shows an example of a user profile 800 in a table format. Various user parameters are listed such as John's height, weight, gait, ethnicity, gender, medications he takes, and thresholds for asthma attacks and high blood pressure.

The user profile 800 may also indicate capabilities a device should use to detect one or more user conditions and/or rates of change. For example, it may indicate that the device should use a device's microphone to detect the user is wheezing to confirm that the user is experiencing an asthma attack.

The device may use the one or more rates of change may be related to the probabilities associated with one or more user conditions. For example, the device may be preconfigured to determine there is a fifty percent chance of a heart attack if there are forty beats per minute decrease within a five minute window.

The device may use the one or more rates of change to extrapolate potential user conditions that may occur if a user continues along with their current behavior. For example, if the user is eating salty food and their heart rate increases twenty beats per minute, the device may determine at the current values the user is not in danger but may extrapolate that if this continues at a similar pace that the user will be in danger within thirty minutes, so it may display or provide a notification so a user can stop their current behavior.

At block 240, the mobile device 100 and/or wearable device 170 may provide one or more messages based on the one or more potential user conditions. The one or more messages may be a notification (e.g. feedback, user prompt, etc.) to the user (visual, auditory, haptic or any combination thereof), it may be one or messages (e.g may be a data message, visual notification, auditory notification, haptic notification, etc)to a third party or a server (e.g. crowdsourcing server, insurance company, OEM, doctor, hospital, emergency services, family member, friend, a person from a contact list, etc.), etc.

The one or more messages may be derived from the user condition, but it does not need to explicitly include the user condition. For example, if the user condition is diabetic reaction then the one or more messages may be feedback that states “Please avoid drinking coffee and consuming salty food”, “Please take your blood pressure medication now”, and/or “Please avoid further sugar or carbs.” These one or more messages responses may be known a-priori or may be dynamically generated. In one embodiment, the one or more messages may indicate the causes of the user symptoms, user conditions, etc; and/or may indicate actions the user can take to resolve the user condition and/or user symptoms.

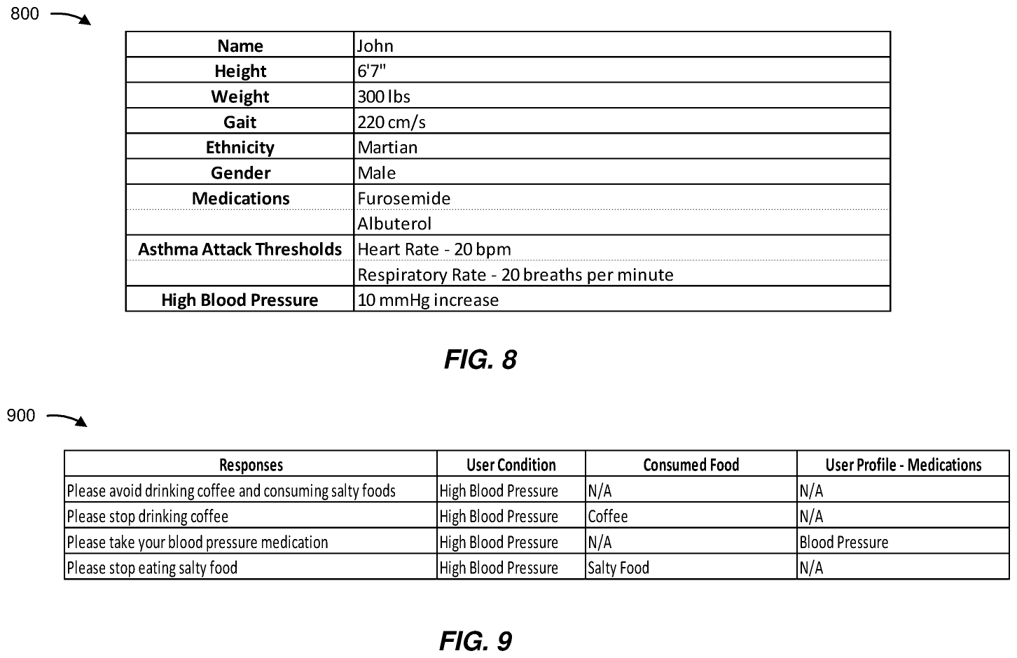

The one or more messages may be pre-associated with various user conditions. For example, the one or more messages may be stored in column of a table with a second column indicating the associated user conditions for that response. FIG. 9 provides an example table 900 for illustrative purposes that shows various responses that may be provided to the user based on the user condition, consumed food, user profile—medications, etc.

The table 900 may also indicate responses that may be dynamic. For example, if the device detected the user is experiencing an asthma attack or high blood pressure, it may determine how to adjust the user's medication to further help the patient, so the response to the user is the adjusted medication dosage, new medication, etc. It may send a request to a doctor for validation, confirmation or changes so the doctor can make the final determination. Along with the request, it may provide the user's physiological parameters and trends of those physiological parameters. The doctor may request additional validation or measure new physiological parameters before the doctor authorizes the additional dosage/medication. This may also be done with a different third party (e.g. insurance company, relative, care giver, etc.) or in addition to the doctor.

In one embodiment, the one or more messages may be a request for information from the user based on the identified one or more potential user conditions. For example, it may ask the user if they have consumed anything recently and if so, what was it that they consumed. These may be text boxes that allow the user to enter in a response and/or it may be a pre-defined selectable response. For example, it may allow the user to type in “Med”, but the selectable response comes up with “Medication” for the user to select.

If the one or more messages is a request for information, then the response to the request for information is received by the mobile device 100 and/or wearable device 170 and the response may indicate one or more user condition in the potential user condition. The device, in response to the user's response(s), may store the one or more responses and the one or more rates of change. According to the aspect of the disclosure, the device may also store the identified user condition(s) that were based on the user's response(s) and the potential user condition(s).

The responses from the user may indicate the user consumed one or more beverages, one or more foods, administered one or more medications or any combination thereof. For example, the user may indicate that the user has consumed or was injected with blood pressure medication.

In some cases, the user may have an adverse side effect from a consumed product and/or medication. In those circumstances, the adverse side effect that is detected from the rates of change for one or more physiological parameters may be reported to a third party, a crowdsourcing server, etc. If the user is unaware of the adverse side effect but this was previously detected by the device and/or by another device (e.g. crowdsourced) then the device can notify the user of the adverse side effect and may even notify them on how to address the side effect and/or alternative consumed products and/or medications.

According to an aspect of the disclosure, the device may determine whether there is an emergency based on the identified one or more potential user conditions. For example, there may be some user conditions that are identified as life threatening and may also indicate one or more thresholds for when emergency services should be contacted. If the one or more rates of changes meet or exceed these emergency threshold(s) then emergency services may be contacted.

According to an aspect of the disclosure, the device may be configured by an OEM, user, doctor, insurance company, crowdsourcing data or any combination thereof to determine whether there is an emergency. For example, a doctor may program the device (directly or remotely) to look for particular issues, such as heart rate increases by fifty beats per minute within a five minute window. The entity configuring the device may also indicate the emergency name (e.g. “possible heart attack”). In one embodiment, the one or more messages may be provided to a third party, such as an OEM, doctor, insurance company, etc.

Once a potential emergency has been identified, the user may be prompted about the emergency for the user to provide additional information, and in some circumstances the emergency may be alternatively or in addition to configured to contact relatives, friends, emergency services, etc. For example, if there is a potential heart attack it may automatically the nearest person on the device contact list or a remote contact list that is associated with the device. It may contact the user's doctor so the doctor can determine whether the user is experiencing a heart attack and the doctor can suggest medications or ways to help if they determine it is or is not a heart attack.

While it is called an emergency, it indicates a follow up is needed, meaning it could be a follow up appointment with a doctor, lab tests need to be performed, etc. In one example, instead of a person being contacted when an emergency-type event is detected then it may automatically schedule the next available doctor's appointment on the user's behalf when the event is detected.

In one embodiment, the device may also check whether the user is conscious. If the user is not conscious then emergency services may automatically be contacted.

The device may check if there were any violations of a court order, doctor order, insurance company order, etc. that may indicate consumption of banned substances (e.g. drugs, alcohol, performance enhancing drugs, etc).

In one embodiment, the device may notify the user that there may be an emergency based on the identified one or more potential user conditions. The user may determine how to response based on the notification. For example, the user may ignore the notification, may request the device to contact emergency services, etc. It is important to note that any notification or prompt discussed throughout the specification may be a visual notification/prompt, auditory notification/prompt, haptic notification/prompt or any combination thereof.

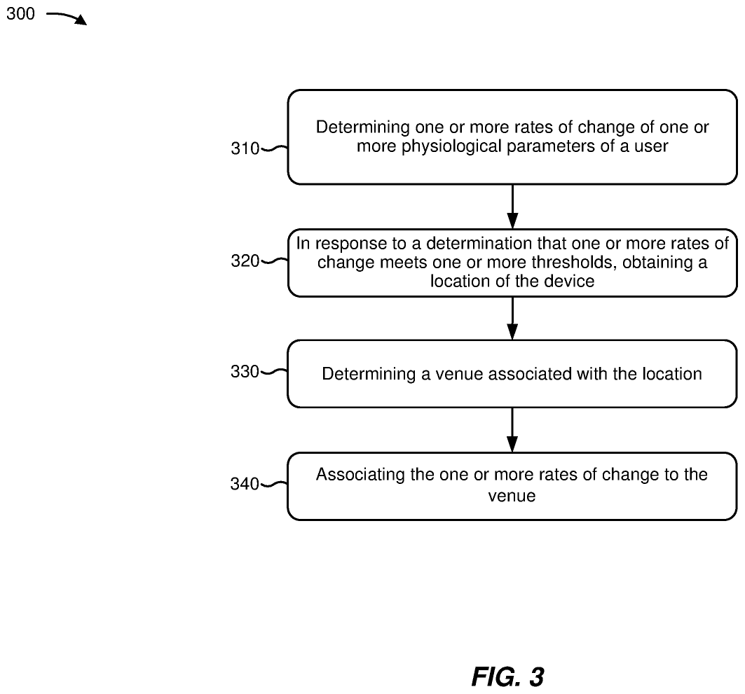

FIG. 3 is a process diagram 300 illustrating an example method of associating one or more rates of change with a venue. This process diagram may be performed by a mobile device 100, wearable device 170 and/or a server 140.

At block 310, the device may determine one or more rates of change of one or more physiological parameters of a user. The description above in block 210 and other parts of the specification are applicable here as well.

At block 320, the device in response to a determination that one or more rates of change meets one or more thresholds, the device obtains a location of the user device. The user device may determine its location via GNSS, WWAN, WLAN, WPAN, Visual Inertial Odometry (VIO), IMU, hybrid approaches or any combination thereof. If the device is not the user device (e.g. mobile device 100 or wearable device 170), then the device may either request the location from the user device or may be automatically provided with the user device's location over a communication link (wireless and/or wired). The description above in block 220 and other parts of the specification are applicable here as well.

In one embodiment, the device may compare the one or more rates of change of one or more physiological parameters to one or more physiological parameter profiles or consumption profile or some other profile (these may also be part of the user profile). If the pattern is already found in the profile or a similar pattern is already in the profile, then there may be an action, user conditions, consumed product that is already associated with that pattern. In one example, the user may have previously been notified of these issues associated with that pattern and dismissed the prompt, so for future notifications that are trigger based on a similar pattern they may be automatically dismissed. If there isn't a high similarity that meets or exceeds a threshold, then it may proceed to block 330.

At block 330, the device may determine one or more venues associated with the location. It may have a point of interest database that indicates a venue associated with a location. Since location has uncertainty associated with it, there may be multiple venues associated with a location when including the uncertainty of the location. The device may identify the one or more venues based on the point of interest databased and the location. The description above in block 230 and other parts of the specification are applicable here as well.

At block 340, the device may associate the one or more rates of change to the venue. This may consist of storing this information, generating a data message that indicates the venue is associated with the one or more rates of change and providing that to a third party, etc.

In one embodiment, the device may use the one or more rates of change to determine one or more thresholds for the venue and may associate and store the one or more thresholds with the one or more venues. The device may use historical information along with the one or more rates of change to establish the one or more thresholds. This information may also be sent to a crowdsource server to identify how rates of changes are affected by the venues. For example, there may be food at the venue that causes large swings in rates of change, total change over time, etc. This may be used by food inspectors to identify potential issues with restaurants and other food venues, but may also be used by other governmental agencies to identify other potential issues (e.g. Central for Disease Control for identify infection breakouts, etc.).

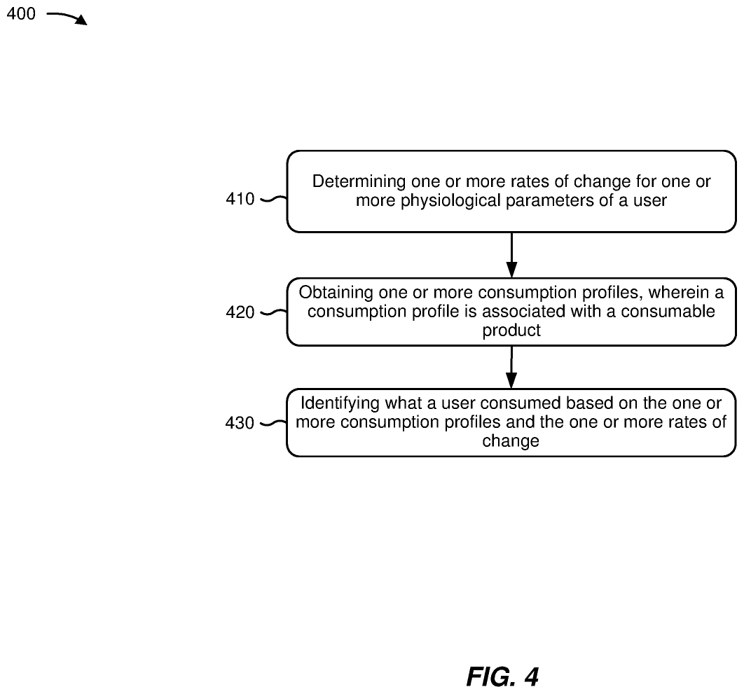

FIG. 4 shows an example process diagram 400 illustrating a method of identifying a consumed product based on changes to a user's physiological parameter(s).

At block 410, a device, such as mobile device 100, wearable device 170 and/or server 140, may determine one or more rates of change for one or more physiological parameters of a user. In one example, the measurements to determine one or more physiological parameters may be determined by the mobile device 100 and/or the wearable 170, but the rates of change may be determined by another entity, such as the mobile device 100 and/or the server 140. It is important to note that the one or more rates of change may be obtained by the mobile device 100 and/or the server 140. The description above in block 210 and other parts of the specification are applicable here as well.

At block 420, a device may obtain one or more consumption profiles, wherein a consumption profile is associated with a consumable product. The consumption profile indicates how one or more physiological parameters of a user may respond based on consuming the consumable product. For example, the user's heart rate may increase ten percentage within fifteen minutes of consuming the product. The consumption profile may indicate which physiological parameter may be affect, how much the physiological parameter may be affected, when and/or how long the physiological parameter may be affected, etc. This may be indicated in absolute values, relative values, one or more rates of change, one or more rates of change within a time window, etc.

According to an aspect of the disclosure, the consumption profile may be tied to a location and/or venue. Once a location and/or venue is determined, the consumption profile may indicate a list of different foods and/or beverages usually available that are associated with the location/venue, so a device can display a pull down or drop down menu for the different foods and/or beverages available so the user can easily select what they are consuming or about to consume. In some cases, prior to ordering a user can determine how the food/beverages will affect the user's physiological parameters and side effects that may occur. For example, if a food is very sugary and the user tends to be tired after the consumption of sugary foods, then the device may provide a notification to the user to indicate that consumption of that particular food will result in the user being tired and if tied to the user's calendar and/or to-do list it may indicate that the user is unlikely to make their appointment or complete the tasks wish to complete today. It is important to note the user may not need to enter in or select this information, but the device can use a microphone to list to the order and provide feedback/notification to the user based on the items they are ordering or discussing.

The consumption profile may be obtained from a third party (e.g. an insurance company, a manufacturer of the consumable product, an independent entity, a regulator or governmental agency, etc). There may be different consumption profiles for the same consumable product, the device may choose which to use, may be configured to use a particular profile or may use a combination of different profiles.

The consumption profile may be in a tabular format, graphical format or both. It may indicate thresholds for how one or more physiological parameters will change within a particular time window, rates of change for one or more physiological parameters, an applicable time window, etc.

In one embodiment, the device may detect and/or identify what a user is about to consume via computer vision (e.g. camera), NFC/RF tags, etc. The device may detect what the user is about to consume and provide one or more messages to the user and/or a third party (e.g. crowdsourcing server, insurance company, OEM, doctor, etc). If this is provided to a third party, the third party can provide a response message to the user indicating to stop consuming the product, what they can do to counteract the consumed product, positive feedback for consuming the product, charge you more for insurance based on going against the instructions, etc.

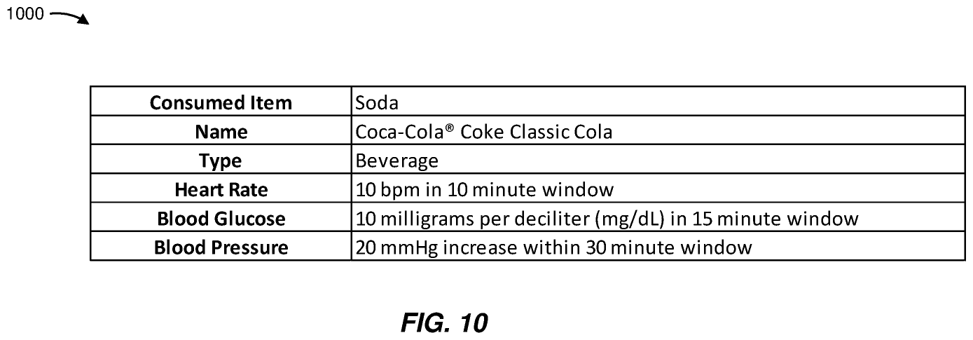

FIG. 10 shows an example table for a consumption profile 1000 for soda. It may indicate the particular name of the soda, type of product, size of the product (e.g. ounces, fluid ounces, etc.) and one or more physiological parameters and how they may be affected. If there are a plurality of physiological parameters these can be used in combination to identify the consumed product or may be used individually or in any combination (e.g. first physiological parameter and third physiological parameter may be used). In this example, if the device determines there has been a ten bpm increase in the last ten minutes, then it may identify that the consumed product was Coca-Cola® Coke Classic Cola or may identify it as a potential consumed product. If it is marked as a potential consumed product, it may wait for the blood glucose and/or the blood pressure to further confirm, and/or it may ask the user if they consumed a soda or specifically a Coca-Cola® Coke Classic Cola.

At block 430, the device may identify what a user consumed based on the one or more consumption profiles and the one or more rates of change. The device may compare the one or more rates of change for the one or more physiological parameters to identify from a plurality of consumption profiles, which consumable product was consumed by the user. For example, if there is a consumption profile for coffee that indicate a heart rate change of ten bpm with a time window of twenty minute and a second consumption profile for asthma medication that indicate a heart rate change of twenty five bpm with a time window for five minutes, then if the rate of change for heart rate was thirty bpm within a time window for four minutes then the device may identify the consumed product as potentially being asthma medication. Since different users may be affected by the consumed product, the consumption profile may need to be adapted over time specifically to the user. The device may jumpstart the process by asking the user basic questions such as gender, height, weight, ethnicity, location, etc. so that the device can obtain a user-similar consumption profile, similar to a user-similar profile as described earlier in the specification.

In one embodiment, the device may obtain what a user has ordered (e.g. restaurant order, grocery order, etc). The device may use the user's order to narrow down what the user may have consumed. The device may use the user's order to preemptively provide one or more messages to the user about what not to consume and/or how the user's body will react to consuming products from that order.

According to an aspect of the disclosure, the device can log the consumed product or store the time/date and characteristics associated with the consumed product (e.g. calories, fat, sodium, etc) so the user may have a holistic view of what they are consuming and how it is affect their one or more physiological parameters. This information may be displayed to the user in a tabular format, graphical format (e.g. charts), etc. This information may be used to notify the user that they have already taken their medication, provide prompts for the user to take their medication, etc.

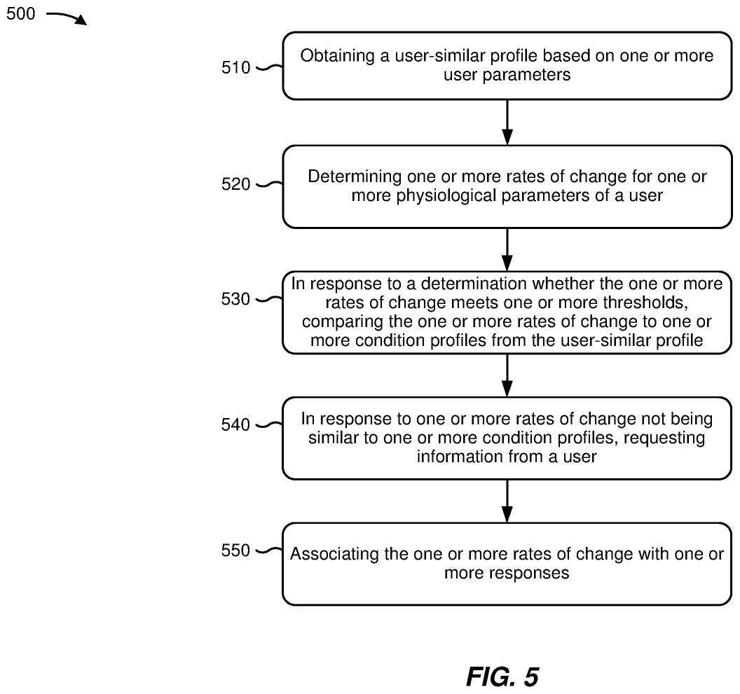

FIG. 5 is an example process diagram illustrating a method of deriving a user-specific profile from a user-similar profile

At block 510, the device, such as a mobile device 100 and/or wearable device 170, may obtain a user-similar profile based on one or more user parameters. A user may enter via the device (e.g. touch screen, keyboard, user interface, voice dictation, etc.) and/or the device may obtain one or more user parameter associated with the user. The user parameters may be build/shape, height, weight, water weight, muscle weight, bone mass, heart rate, blood pressure, other physiological parameters of the user, ethnicity, age, gender, or any combination thereof.

The user parameters may be used to obtain a profile of a similar user or a profile of a group of similar users. For example, this may be anonymized data that indicates user parameters that enables another user with similar parameters or characteristics to identify potential user conditions.

At block 520, the device may determine one or more rates of change for one or more physiological parameters of a user. The description above in block 220 and other parts of the specification are applicable here as well. It is important to note that a mobile device 100 and/or server 140 may perform this process 500 even if the measurements are performed on another device, such as the wearable device 170, but the mobile device 100 and/or the server 140 would obtain the one or more rates of change or may obtain the physiological parameters over a period of time and it could determine the one or more rates of change.

At block 530, in response to a determination whether the one or more rates of change meets one or more thresholds, the device compares the one or more rates of change to one or more condition profiles from the user-similar profile.

At block 540, if the device does not determine there is a similarity to one or more condition profiles, if the device wants to improve confidence in its determination or it may be programmed to request this information, the device may request information from the user. The information request (as described through the specification) may ask if the user is experiencing a condition, if they consumed a product (e.g. food, beverage, medication, etc), time of consumption, if they were exercising, etc.

At block 550, the device associates the one or more rates of change with one or more responses. This may be associated in a user-specific profile. This may be storing the information on the device, providing this associated with another entity (e.g. mobile device 100 and/or server 140), etc. For example, if the response is drinking soda then the rate of change is associated with drinking soda and stored. This information may be used later on by the device to identify what the user has consumed.

This process, along with the processes described throughout the specification, are beneficial, because it enables a user to receive notifications about potential user conditions, enables a user to automatically log consumption of beverages, food, medication, time of consumption, etc., enables a device to determine user-specific differences from other user-similar profiles, etc.

Over time, the user-similar profile may be adapted with user-specific threshold(s) so that the user's sensitivity for one or more physiological parameters are determined for rates of change for user conditions and/or symptoms and/or for consumed product(s).

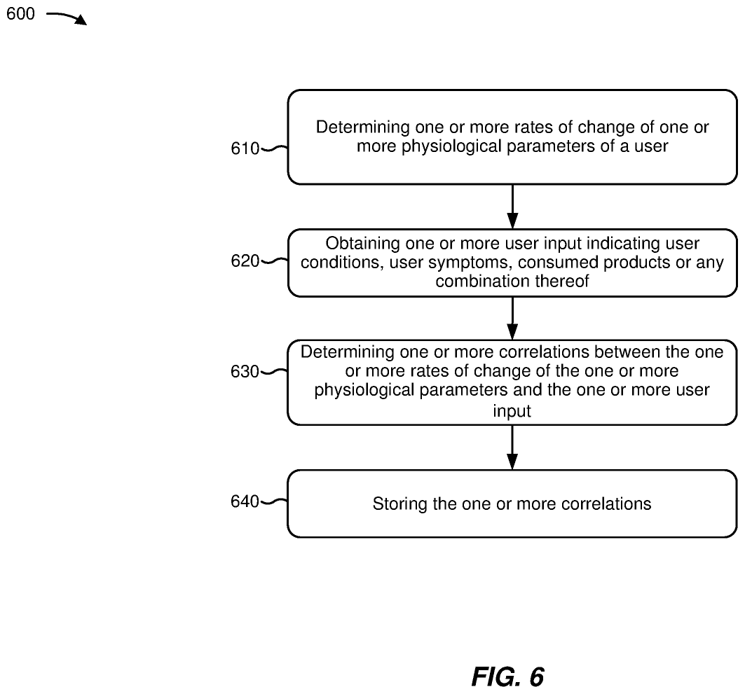

FIG. 6 shows an example process diagram illustrating a method of associating physiological parameters and user conditions.

At block 610, a device determines one or more rates of change of one or more physiological parameters of a user. The description above in block 210 and other parts of the specification are applicable here as well.

At block 620, the device obtains one or more user input indicating user conditions, user symptoms, consumed products or any combination thereof. The user may log this information on the device, on a remote device (e.g. server) or both. If the user logs this information into a remote device, the device may obtain this information through a wired or wireless communication link. For example, the user may log this information onto a website through a personal computer, the website may store this information and the device may retrieve this information or be sent this information from the website server.

The user input may be unsolicited by the application(s), operating system, and/or the device. In one embodiment, the user may have a prompt that is always available on the device for them to log the user conditions, symptoms, consumed products, etc. According to an aspect of the disclosure, the user may open an application to log this information.

The application and/or operating system that is performing block 610 may or may not be the same application where the user is logging this information. For example, the user may log this information using a first application, but the rates of change of physiological parameter may be a second application (or the operating system or dedicated functionality of the device), but the information of the two application may be shared (e.g. one way from the first application to the second application (or vice versa), or it may be bi-directional sharing).

The user may indicate the time of the conditions, symptoms, consumed products, etc. In one embodiment, the application/operating system may automatically log the time. For example, if the user inputted that they ate pizza, the application can include a timestamp of when the log was entered.

The user may indicate their confidence in the conditions and/or symptoms. They may indicate that the confidence level is low, medium, high, etc. It may also be numerical based.

The user may indicate brand information and/or other specific information about the consumed product. For example, if it is a food product they may indicate calories, fat, salt, etc. If the product is medication, they may indicate the medication name, dosage, etc.

The user may also enter in doctor's instructions for medication. For example, they may indicate they need to take blood pressure medication twice a day (once in the morning and another in the evening). As described throughout the specification, this may be used to determine compliance with medication and indicate whether they have already taken the medication and when they still need to take the medication.

In one embodiment, the user may indicate whether the conditions, symptoms, and/or consumed product is quasi-periodically recurring. For example, they may indicate they have recurring migraines in the afternoon.

In one embodiment, the user may also log activity and intensity of the activity. This may also be automatically logged by the device. In one embodiment, the user may indicate additional information or more specifically indicate what kind of activity was performed (e.g. indoor exercise bike).

At block 630, the device may determine one or more correlations between one or more rates of change of the one or more physiological parameters and the one or more user inputs.

The device may look at different time periods starting from when the user input indicates (e.g. headache started an hour ago) and determine how the one or more rates of change have changed during one or more time periods. For example, the user's blood pressure may have spiked an hour before the headache started, and fifteen minutes prior the user may have logged they consumed a cup of coffee so a correlation may be determined indicating the user may experience headaches when they are consumed coffee. In cases where the device attempts to prevent this condition, they have to notify the user that they should consume water in addition to the coffee (or preemptively take pain medication).

In one embodiment, a time delay may also be determined from when the user consumed the product (e.g. started consuming, finishing consuming, etc) and when the physiological response is detected by the device. The device may detect when the user is consuming a product in various ways such as inertial measurement unit (e.g. motion data), computer vision, NFC/RF Tags, acoustics or any combination thereof.

The device may split the time periods into smaller chunks of time (e.g. if original time period is milliseconds, the smaller chunks of time may be a factor of 10 or more lower such microseconds or picoseconds) as and compare how the rates of change for one or more physiological parameters have changed for a user over different instances with similar conditions/symptoms/consumed products. For example, if on Monday, the user experienced a headache but first consumed coffee (elevated blood pressure) and then exercised (elevated heart rate, excessive sweating) prior to experiencing a headache, but on Tuesday the user only experienced excessive sweating without exercising or drinking coffee, then the device may determine that dehydration is causing the user's headache.

In one embodiment, the correlation(s) may be sent to a health server and/or a crowdsourcing server to verify if the correlations are correct or need to be validated. If the server determines the correlation appears to be unique compared to the other users then it may indicate the correlation has a low confidence level or may require further validation over a long period of time (e.g. days, weeks, years). If the server determines other users have similar correlations then it may have a high confidence level and use the correlation as part of the crowdsourcing database on a crowdsourcing server.

At block 640, the device may store the correlation information locally at the device, at a remote device (e.g. server) or both.

In one embodiment, the device may provide anonymized information for the server so other devices may use this information to identify correlations among a larger group of people. For example, users of a particular medication may indicate how their rates of changes for one or more physiological parameters are changing after taking the medication and/or some time period after the medication. This may be used to determine how likely it may be to experience the side effects of the medication, it may be used to determine whether there is a new issue/side effect that users are experiencing, etc. This may similarly be done to identify user conditions, symptoms, other consumed products (e.g. food, beverages, etc). or any combination thereof.

As described throughout the specification, the device may transmit the correlation, one or more rates of change for the one or more physiological parameters, total change over a period of time for one or more physiological parameters, absolute values for one or more physiological parameters or any combination thereof to a crowdsourcing server. This information may be anonymized, but it may also include general user parameters so a user-similar profile may be generated. For example, device may transmit the following user parameters to a crowdsource server: gender, ethnicity, age, weight, height, known health conditions or any other user parameters or any combination thereof. It may also provide the trends of the rates of change (and other parameters) over a period of time and not just when the user condition is detected but how the user's body responds over a longer period of time after the detection (e.g. hours after detection, days after detection, weeks, years, etc).

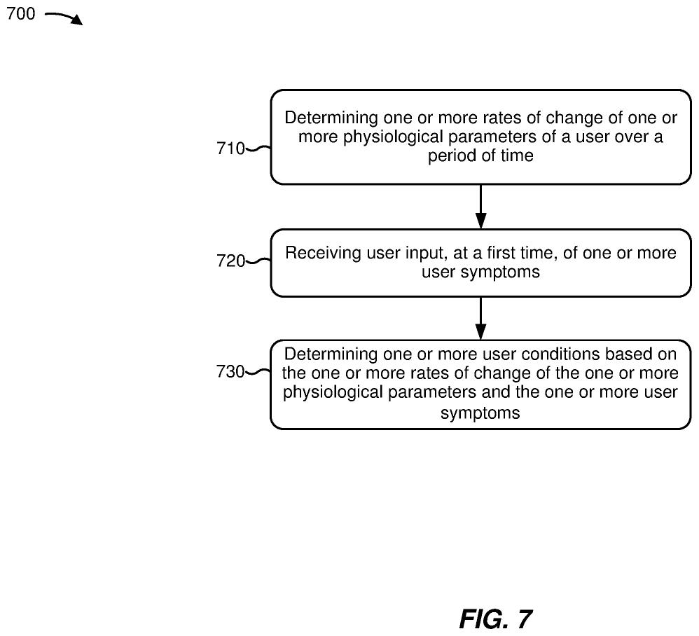

FIG. 7 shows an example process diagram illustrating a method of determining a user condition.

At block 710, a device determines one or more rates of change of one or more physiological parameters of a user over a period of time. The description above in block 210 and other parts of the specification are applicable here as well. The period of time may be minutes, hours, days, weeks, months, years, etc, but it is not instantaneous and it is not over a few seconds.

At block 720, a device receives user input, at a first time, of one or more user symptoms. The user input may be logged into a first application, operating system or dedicated functionality of the device. This may be unsolicited information or may be requested by the application, operating system and/or dedicated functionality. The user input may be a selection from a list of potential user symptoms or may be entered by the user. The description above in block 620 and other parts of the specification are applicable here as well.

The user input may indicate a time when the symptom started, the symptom ended, duration, intensity, pattern, location on the user, location of the incident, or any combination thereof.

The device may, either in addition to or alternative to user input, identify an activity and/or location and determine a user context in which to evaluate the one or more rates of change of one or more physiological parameters.

At block 730, the device determines one or more user condition based on the one or more rates of changes of the one or more physiological parameters and the one or more user symptoms.

In one embodiment, the device may have a lookup table of historical data, preconfigured data, OEM data, etc. The device may use the lookup table to determine one or more user conditions based on the lookup table, one or more rates of changes of the one or more physiological parameters and the one or more user symptoms.

According to an aspect of the disclosure, the device may receive crowdsourced information that indicates how the rates of changes, user symptoms and user conditions are correlated. The device may determine the user condition(s) based on the crowdsourced information, rates of change and the user symptoms.

Any of the processes, embodiments, etc. as described throughout the specification are merely examples and may be combined and/or modified in various ways. For example, portions of FIG. 2 may be combined with portions of FIG. 7.

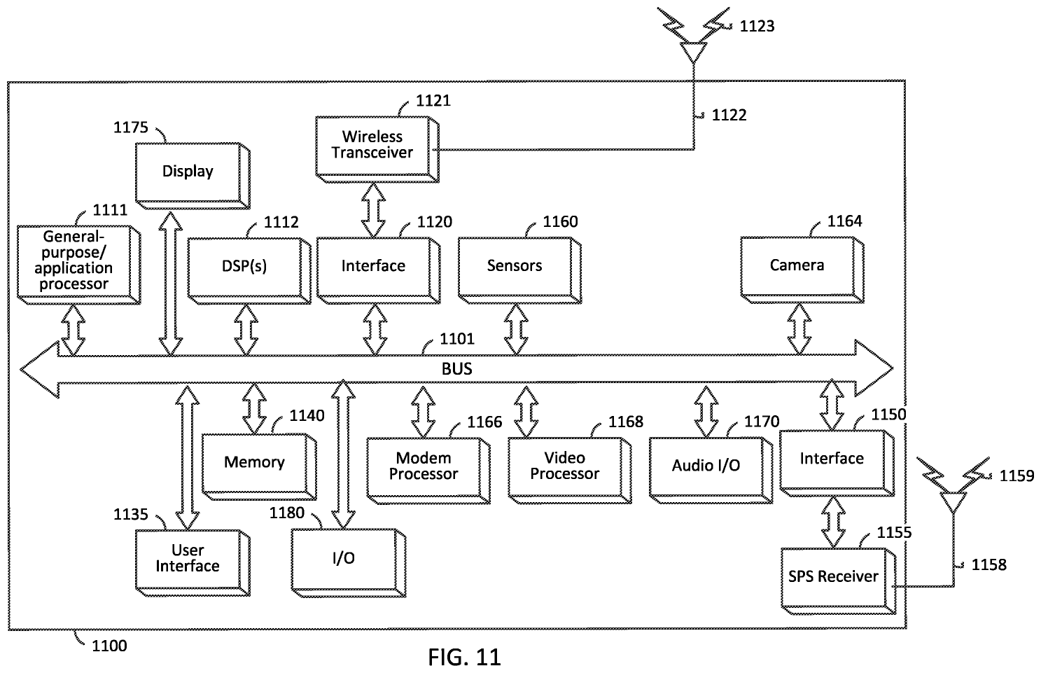

FIG. 11 is a schematic diagram of a device 1100 according to an implementation. Mobile device 100 shown in FIG. 1 may comprise one or more features of device 1100 shown in FIG. 11, and similarly wearable device 170 shown in FIG. 1 may comprise one or more features of the device 1100. In certain implementations, device 1100 may comprise a wireless transceiver 1121 which is capable of transmitting and receiving wireless signals 1123 via wireless antenna 1122 over a wireless communication network. Wireless transceiver 1121 may be connected to bus 1101 by a wireless transceiver bus interface 1120. Wireless transceiver bus interface 1120 may, in some implementations be at least partially integrated with wireless transceiver 1121. Some implementations may include multiple wireless transceivers 1121 and wireless antennas 1122 to enable transmitting and/or receiving signals according to corresponding multiple wireless communication standards such as, for example, versions of IEEE Standard 802.11, CDMA, WCDMA, LTE, UMTS, GSM, AMPS, Zigbee, Bluetooth and a 5G or NR radio interface defined by 3GPP, just to name a few examples. In a particular implementation, wireless transceiver 1121 may transmit signals on an uplink channel and receive signals on a downlink channel as discussed above. It is important to note that transceiver used here can also mean one or more discrete transmitters and/or one or more discrete receivers.

Device 1100 may also comprise SPS receiver 1155 capable of receiving and acquiring SPS signals 1159 via SPS antenna 1158 (which may be integrated with antenna 1122 in some implementations). SPS receiver 1155 may also process, in whole or in part, acquired SPS signals 1159 for estimating a location of device 1100. In some implementations, general-purpose processor(s) 1111, memory 1140, digital signal processor(s) (DSP(s)) 1112 and/or specialized processors (not shown) may also be utilized to process acquired SPS signals, in whole or in part, and/or calculate an estimated location of device 1100, in conjunction with SPS receiver 1155. Storage of SPS or other signals (e.g., signals acquired from wireless transceiver 1121) or storage of measurements of these signals for use in performing positioning operations may be performed in memory 1140 or registers (not shown). General-purpose processor(s) 1111, memory 1140, DSP(s) 1112 and/or specialized processors may provide or support a location engine for use in processing measurements to estimate a location of device 1100. In a particular implementation, all or portions of actions or operations set forth for process 200, 300, 400, 500, 600 and/or 700 may be executed by general-purpose processor(s) 1111 or DSP(s) 1112 based on machine-readable instructions stored in memory 1140.

Also shown in FIG. 11, digital signal processor(s) (DSP(s)) 1112 and general-purpose processor(s) 1111 may be connected to memory 1140 through bus 1101. A particular bus interface (not shown) may be integrated with the DSP(s) 1112, general-purpose processor(s) 1111 and memory 1140. In various implementations, functions may be performed in response to execution of one or more machine-readable instructions stored in memory 1140 such as on a computer-readable storage medium, such as RAM, ROM, FLASH, or disc drive, just to name a few examples. The one or more instructions may be executable by general-purpose processor(s) 1111, specialized processors, graphics processing unit(s) (GPU), neural processor(s) (NPU), AI accelerator(s), or DSP(s) 1112. Memory 1140 may comprise a non-transitory processor-readable memory and/or a computer-readable memory that stores software code (programming code, instructions, etc.) that are executable by processor(s) 1111 and/or DSP(s) 1112. The processor(s) 1111 and/or the DSP(s) 1112 may be used to perform various operations as described throughout the specification.

Also shown in FIG. 11, a user interface 1135 may comprise any one of several devices such as, for example, a speaker, microphone, display device, vibration device, keyboard, touch screen, just to name a few examples. In a particular implementation, user interface 1135 may enable a user to interact with one or more applications hosted on device 1100. For example, devices of user interface 1135 may store analog or digital signals on memory 1140 to be further processed by DSP(s) 1112 or general-purpose processor 1111 in response to action from a user. Similarly, applications hosted on device 1100 may store analog or digital signals on memory 1140 to present an output signal to a user. In another implementation, device 1100 may optionally include a dedicated audio input/output (I/O) device 1170 comprising, for example, a dedicated speaker, microphone, digital to analog circuitry, analog to digital circuitry, amplifiers and/or gain control. Audio I/O 1170 may also include ultrasound or any audio-based positioning that can be used to determine the position, orientation or context of the device 1100. Audio I/O 1170 may also be used to provide data via one or more audio signals to another source. It should be understood, however, that this is merely an example of how an audio I/O may be implemented in a mobile device, and that claimed subject matter is not limited in this respect.

Device 1100 may also comprise a dedicated camera device 1164 for capturing still or moving imagery. Camera device 1164 may comprise, for example an imaging sensor (e.g., charge coupled device or CMOS imager), lens, analog to digital circuitry, frame buffers, just to name a few examples. In one embodiment, additional processing, conditioning, encoding or compression of signals representing captured images may be performed at general purpose/application processor 1111 or DSP(s) 1112. Alternatively, a dedicated video processor 1168 may perform conditioning, encoding, compression or manipulation of signals representing captured images. Additionally, video processor 1168 may decode/decompress stored image data for presentation on a display device (not shown) on device 1100. The video processor 1168, may be an image sensor processor, and may be capable of performing computer vision operations.

Camera device 1164 may include image sensors. The image sensors may include cameras, charge coupled device (CCD) based devices, or Complementary Metal Oxide Semiconductor (CMOS) based devices, Lidar, computer vision devices, etc. on a vehicle, which may be used to obtain images of an environment around the vehicle. Image sensors, which may be still and/or video cameras, may capture a series of 2-Dimensional (2D) still and/or video image frames of an environment. In some embodiments, image sensors may take the form of a depth sensing camera, or may be coupled to depth sensors. The term “depth sensor” is used to refer to functional units that may be used to obtain depth information. In some embodiments, image sensors 232 may comprise Red-Green-Blue-Depth (RGBD) cameras, which may capture per-pixel depth (D) information when the depth sensor is enabled, in addition to color (RGB) images. In one embodiment, depth information may be obtained from stereo sensors such as a combination of an infra-red structured light projector and an infra-red camera registered to a RGB camera. In some embodiments, image sensors may be stereoscopic cameras capable of capturing 3 Dimensional (3D) images. For example, a depth sensor may form part of a passive stereo vision sensor, which may use two or more cameras to obtain depth information for a scene. The pixel coordinates of points common to both cameras in a captured scene may be used along with camera parameter information, camera pose information and/or triangulation techniques to obtain per-pixel depth information. In some embodiment, the image sensor may be capable of capturing infrared or other non-visible light (i.e. not visible to the human eye). In some embodiments, image sensor may include Lidar sensors, which may provide measurements to estimate the relative distance of objects. The camera 1164 may also be capable of receiving visual light communication data by capturing optical measurements and demodulating to receive this data. The term “camera pose” or “image sensor pose” is also used to refer to the position and orientation of an image sensor on a subject vehicle.

Device 1100 may also comprise sensors 1160 coupled to bus 1101 which may include, for example, inertial sensors and environment sensors. Inertial sensors of sensors 1160 may comprise, for example accelerometers (e.g., collectively responding to acceleration of device 1100 in three dimensions), one or more gyroscopes or one or more magnetometers (e.g., to support one or more compass applications). Environment sensors of device 1100 may comprise, for example, temperature sensors, barometric pressure sensors, ambient light sensors, camera imagers, microphones, just to name few examples. The sensors 1160 may include biometric sensors. The biometric sensors may comprise photoplethysmography (PPG), electrocardiography (ECG), peripheral capillary oxygen saturation (SpO2), blood pressure and/or pulse transit time sensor, body temperature sensor, ultrasonic sensor/acoustic sensor, pressure sensor, heart rate sensor, respiration rate sensor, electrodermal activity (EDA), inertial motion unit sensors (IMU), photoacoustic sensors, or any combination thereof. Sensors 1160 may generate analog or digital signals that may be stored in memory 1140 and processed by DSP(s) 1112 or general-purpose application processor 1111 in support of one or more applications such as, for example, applications directed to positioning or navigation operations. The sensors 1160 may also include radar 1162, which may be used to determine the distance between the device and another object. The sensors 1160, SPS receiver 1155, wireless transceiver 1121, camera(s) 1164, audio i/o 1170, radar, ultrasound, or any combination thereof may be used determine one or more location measurements and/or a position location of the device 1100.

Device 1100 may comprise one or more displays 1175 and/or one or more display controllers (not shown). The displays 1175 and/or the display controllers may provide and/or display a user interface, visual alerts, metrics, and/or other visualizations. In one embodiment, the one or more displays 1175 and/or display controllers may be integrated with the device 1100.

According to another aspect of the disclosure, the one or more displays 1175 and/or display controllers may be external to the device 1100. The device 1100 may have one or more input and/or output ports (I/O) 1180, through a wired or wireless interface, and may use the I/O to provide data to the external one or more displays 1175 and/or display controller(s).

The I/O 1180 may be used for other purposes as well, such as but not limited to obtaining data from a vehicle's onboard diagnostics, vehicle sensors, providing sensor information from the device 1100 to the external device, etc. The I/O 1180 may be used to provide data, such as position information, to another processor and/or component, such as the behavior and/or route planning component 1190.

The general purpose/application processor 1111, DSP 1112, GPU, neural processor(s) (NPU), AI accelerators, microcontrollers, controllers, video processor(s) 1168, memory 1140, sensors 1160, camera 1164, wireless transceiver 1121, SPS receiver 1155, audio i/o 1170, user interface 1135, display 1175, haptic engine, modem processor 1166 may be used to perform one or more steps in FIG. 2, FIG. 3, FIG. 4, FIG. 5, FIG. 6 and/or other parts of the specification. For example, general purpose/application processor 1111, DSP 1112, GPU, neural processor(s) (NPU), AI accelerators, microcontrollers, controllers, video processor(s) 1168, memory 1140, sensors 1160, camera 1164, or audio i/o 1170 or any combination thereof may be used in the process described in blocks 210, 220, 230, 310, 320, 410, 420, 430, 520, 530, 540, 550, 610, 620, 630, 640, 710, 720 and/or 730. In another example, general purpose/application processor 1111, DSP 1112, GPU, neural processor(s) (NPU), AI accelerators, microcontrollers, controllers, video processor(s) 1168, memory 1140, sensors 1160, camera 1164, wireless transceiver 1121, SPS receiver 1155, audio i/o 1170, user interface 1135, display 1175, haptic engine, modem processor 1166 or any combination thereof may be used in the process described in blocks 230, 240, 330, 340, 420, 430, 530, 540, 550, 610, 620, 630, 640, 710, 720 and/or 730.

In a particular implementation, device 1100 may comprise a dedicated modem processor 1166 capable of performing baseband processing of signals received and down converted at wireless transceiver 1121 or SPS receiver 1155. Similarly, modem processor 1166 may perform baseband processing of signals to be upconverted for transmission by wireless transceiver 1121. In alternative implementations, instead of having a dedicated modem processor, baseband processing may be performed by a general-purpose processor or DSP (e.g., general purpose/application processor 1111 or DSP(s) 1112). It should be understood, however, that these are merely examples of structures that may perform baseband processing, and that claimed subject matter is not limited in this respect.

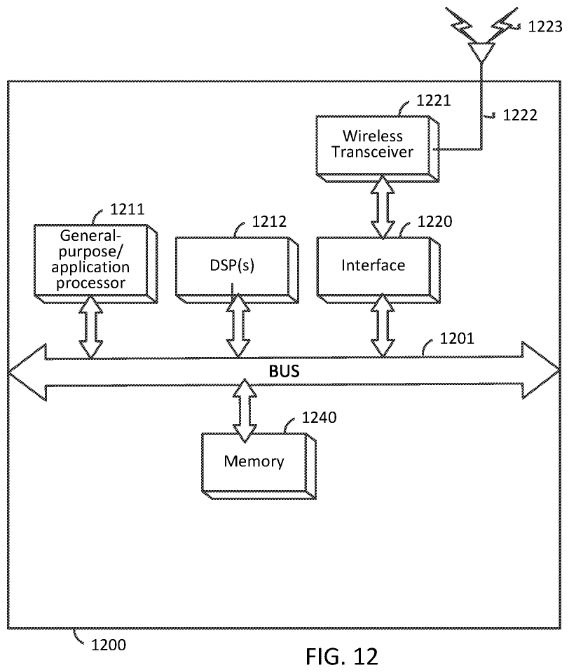

FIG. 12 is a schematic diagram of a server 1200 according to an implementation. Server 140 shown in FIG. 1 may comprise one or more features of server 1200 shown in FIG. 12. In certain implementations, server 1200 may comprise a wireless transceiver 1221 which is capable of transmitting and receiving wireless signals 1223 via wireless antenna 1222 over a wireless communication network. Wireless transceiver 1221 may be connected to bus 1201 by a wireless transceiver bus interface 1220. Wireless transceiver bus interface 1220 may, in some implementations be at least partially integrated with wireless transceiver 1221. Some implementations may include multiple wireless transceivers 1221 and wireless antennas 1222 to enable transmitting and/or receiving signals according to corresponding multiple wireless communication standards such as, for example, versions of IEEE Standard 802.11, CDMA, WCDMA, LTE, UMTS, GSM, AMPS, Zigbee, Bluetooth and a 5G or NR radio interface defined by 3GPP, just to name a few examples. In a particular implementation, wireless transceiver 1221 may transmit signals on an uplink channel and receive signals on a downlink channel as discussed above.

It is important to note that even if not explicitly mentioned, any of the embodiments or examples described in the specification may use or be implemented with various machine learning/deep learning approaches.

The server 1200 may include a wired interface (not shown in FIG. 12), such as ethernet, coaxial cable, etc.

Also shown in FIG. 12, digital signal processor(s) (DSP(s)) 1212 and general-purpose processor(s) 1211 may be connected to memory 1240 through bus 1201. A particular bus interface (not shown) may be integrated with the DSP(s) 1212, general-purpose processor(s) 1211 and memory 1240. In various implementations, functions may be performed in response to execution of one or more machine-readable instructions stored in memory 1240 such as on a computer-readable storage medium, such as RAM, ROM, FLASH, or disc drive, just to name a few examples. The one or more instructions may be executable by general-purpose processor(s) 1211, specialized processors, or DSP(s) 1212. Memory 1240 may comprise a non-transitory processor-readable memory and/or a computer-readable memory that stores software code (programming code, instructions, etc.) that are executable by processor(s) 1211 and/or DSP(s) 1212. The processor(s) 1211, specialized processor(s), graphics processing unit(s) (GPU), neural processor(s) (NPU), AI accelerator(s), microcontroller(s), controller(s) and/or the DSP(s) 1212 may be used to perform various operations as described throughout the specification.

The general purpose/application processor 1211, DSP 1212, GPU, neural processor(s) (NPU), AI accelerator(s), microcontrollers, controllers, video processor(s), memory 1240 or any combination thereof may perform one or more steps similar to those listed in FIG. 2, FIG. 3, FIG. 4, FIG. 5, FIG. 6, FIG. 7 and/or other parts of the specification. For example, general purpose/application processor 1211, DSP 1212, GPU, neural processor(s) (NPU), AI accelerators, microcontrollers, controllers, video processor(s), memory 1240 or any combination thereof may be used in block 210, 220, 230, 310, 320, 410, 420, 430, 520, 530, 540, 550, 610, 620, 630, 640, 710, 720 and/or 730. In another example, general purpose/application processor 1211, DSP 1212, GPU, neural processor(s) (NPU), AI accelerators, microcontrollers, controllers, video processor(s), memory 1240, wireless transceiver 1221, wired interface (e.g. ethernet), modem processor or any combination thereof may be used in blocks 230, 240, 330, 340, 420, 430, 530, 540, 550, 620, 630, 640, 720 and/or 730.

Discussions of coupling between components in this specification do not require the components to be directly coupled. These components may be coupled directly or through one or more intermediaries. Additionally, coupling does not require they be directly attached, but it may also include electrically coupled, optically coupled, communicatively coupled or any combination thereof.

Reference throughout this specification to “one example”, “an example”, “certain examples”, or “exemplary implementation” means that a particular feature, structure, or characteristic described in connection with the feature and/or example may be included in at least one feature and/or example of claimed subject matter. Thus, the appearances of the phrase “in one example”, “an example”, “in certain examples” or “in certain implementations” or other like phrases in various places throughout this specification are not necessarily all referring to the same feature, example, and/or limitation. Furthermore, the particular features, structures, or characteristics may be combined in one or more examples and/or features.

Some portions of the detailed description included herein are presented in terms of algorithms or symbolic representations of operations on binary digital signals stored within a memory of a specific apparatus or special purpose computing device or platform. In the context of this particular specification, the term specific apparatus or the like includes a general-purpose computer once it is programmed to perform particular operations pursuant to instructions from program software. Algorithmic descriptions or symbolic representations are examples of techniques used by those of ordinary skill in the signal processing or related arts to convey the substance of their work to others skilled in the art. An algorithm is here, and generally, is considered to be a self-consistent sequence of operations or similar signal processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals, or the like. It should be understood, however, that all of these or similar terms are to be associated with appropriate physical quantities and are merely convenient labels. Unless specifically stated otherwise, as apparent from the discussion herein, it is appreciated that throughout this specification discussions utilizing terms such as “processing,” “computing,” “calculating,” “determining” or the like refer to actions or processes of a specific apparatus, such as a special purpose computer, special purpose computing apparatus or a similar special purpose electronic computing device. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device.

In another aspect, as previously mentioned, a wireless transmitter or access point may comprise a cellular transceiver device, utilized to extend cellular telephone service into a business or home. In such an implementation, one or more mobile devices may communicate with a cellular transceiver device via a code division multiple access (“CDMA”) cellular communication protocol, for example.

Techniques described herein may be used with an SPS that includes any one of several GNSS and/or combinations of GNSS. Furthermore, such techniques may be used with positioning systems that utilize terrestrial transmitters acting as “pseudolites”, or a combination of SVs and such terrestrial transmitters. Terrestrial transmitters may, for example, include ground-based transmitters that broadcast a PN code or other ranging code (e.g., similar to a GPS or CDMA cellular signal). Such a transmitter may be assigned a unique PN code so as to permit identification by a remote receiver. Terrestrial transmitters may be useful, for example, to augment an SPS in situations where SPS signals from an orbiting SV might be unavailable, such as in tunnels, mines, buildings, urban canyons or other enclosed areas. Another implementation of pseudolites is known as radio-beacons. The term “SV”, as used herein, is intended to include terrestrial transmitters acting as pseudolites, equivalents of pseudolites, and possibly others. The terms “SPS signals” and/or “SV signals”, as used herein, is intended to include SPS-like signals from terrestrial transmitters, including terrestrial transmitters acting as pseudolites or equivalents of pseudolites.

In the preceding detailed description, numerous specific details have been set forth to provide a thorough understanding of claimed subject matter. However, it will be understood by those skilled in the art that claimed subject matter may be practiced without these specific details. In other instances, methods and apparatuses that would be known by one of ordinary skill have not been described in detail so as not to obscure claimed subject matter.

The terms, “and”, “or”, and “and/or” as used herein may include a variety of meanings that also are expected to depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein may be used to describe any feature, structure, or characteristic in the singular or may be used to describe a plurality or some other combination of features, structures or characteristics. Though, it should be noted that this is merely an illustrative example and claimed subject matter is not limited to this example.

While there has been illustrated and described what are presently considered to be example features, it will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein.

Therefore, it is intended that claimed subject matter is not limited to the particular examples disclosed, but that such claimed subject matter may also include all aspects falling within the scope of appended claims, and equivalents thereof.

For an implementation involving firmware and/or software, the methodologies may be implemented with modules (e.g., procedures, functions, and so on) that perform the functions described herein. Any machine-readable medium tangibly embodying instructions may be used in implementing the methodologies described herein. For example, software codes may be stored in a memory and executed by a processor unit. Memory may be implemented within the processor unit or external to the processor unit. As used herein the term “memory” refers to any type of long term, short term, volatile, nonvolatile, or other memory and is not to be limited to any particular type of memory or number of memories, or type of media upon which memory is stored.

If implemented in firmware and/or software, the functions may be stored as one or more instructions or code on a computer-readable storage medium. Examples include computer-readable media encoded with a data structure and computer-readable media encoded with a computer program. Computer-readable media includes physical computer storage media. A storage medium may be any available medium that can be accessed by a computer. By way of example, and not limitation, such computer-readable media can comprise RAM, ROM, EEPROM, CD-ROM or other optical disk storage, magnetic disk storage, semiconductor storage, or other storage devices, or any other medium that can be used to store desired program code in the form of instructions or data structures and that can be accessed by a computer; disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media.

In addition to storage on computer-readable storage medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims. That is, the communication apparatus includes transmission media with signals indicative of information to perform disclosed functions. At a first time, the transmission media included in the communication apparatus may include a first portion of the information to perform the disclosed functions, while at a second time the transmission media included in the communication apparatus may include a second portion of the information to perform the disclosed functions.

What is claimed is:

1. A method of providing health assistance information from a mobile device, the method comprising:
determining, at the mobile device, one or more rates of change for one or more physiological parameters of a user;
determining, at the mobile device, whether the one or more rates of change meets one or more thresholds;
in response to the determination that the one or more rates of change meets one or more thresholds, identifying, at the mobile device, one or more potential user conditions based on the one or more rates of change, a location of the mobile device and user profile; and
providing one or more messages based on the one or more potential user conditions.

2. The method of claim 1, further comprising:
determining whether there is an emergency based on the identified one or more potential user conditions;
in response to the determination that there is an emergency, providing a notification of the emergency based on the identified one or more potential user conditions.

3. The method of claim 1, wherein the one or more physiological parameters comprises blood pressure, heart rate, glucose, respiratory rate, peripheral capillary oxygen saturation, pulse transit time, stroke volume, cardiac output, muscle contraction, heart rhythm, body temperature, skin temperature, triglycerides, skin conductivity, or any combination thereof.

4. The method of claim 1, wherein the identifying one or more potential user conditions based on the one or more rates of change, the location of the mobile device and the user profile comprises:
identifying a venue associated with the location of the mobile device;
determining whether the one or more rates of change correspond to one or more conditions related to the venue;
in response to the determination that the one or more rates of change are do not correspond to one or more conditions related to the venue, determining one or more potential user conditions based on the one or more rates of change and the user profile; and
associating the one or more potential user conditions with the venue.

5. The method of claim 1, wherein the one or more potential user conditions comprise diabetic reaction, choking, allergic reaction, asthma attack, hypertension, hypoglycemia, atrial fibrillation, tachycardia, a medication controlled medical condition, or any combination thereof.

6. The method of claim 1, wherein the one or more thresholds are based on a user-specific baseline, historical patterns of the one or more physiological parameters or any combination thereof.

7. The method of claim 1, wherein the providing one or more messages based on the identified one or more potential user conditions comprises:
requesting information based on the identified one or more potential user conditions;
receiving one or more responses to the information request;
in response to the one or more responses indicating a user condition other than the one or more potential user conditions, storing the one or more responses and the one or more rates of change.

8. The method of claim 7, wherein the one or more responses comprises a user consumed one or more beverages, consumed one or more foods, administered one or more medications, time of consumption, or any combination thereof.

9. A device for providing health assistance information, the device comprising:
one or more sensors;
one or more memory;
one or more processors communicatively coupled to the one or more memory and the one or more sensors, the one or more processors configured to:
determine, via the one or more sensors, one or more rates of change for one or more physiological parameters of a user;
determine whether the one or more rates of change meets one or more thresholds;
in response to the determination that the one or more rates of change meets one or more thresholds, identify one or more potential user conditions based on the one or more rates of change, a location of the device and user profile; and
provide one or more messages based on the one or more potential user conditions.

10. The device of claim 9, wherein the one or more processors are further configured to:
determine whether there is an emergency based on the identified one or more potential user conditions;
in response to the determination that there is an emergency, providing a notification of the emergency based on the identified one or more potential user conditions.

11. The device of claim 9, wherein the one or more physiological parameters comprises blood pressure, heart rate, glucose, respiratory rate, peripheral capillary oxygen saturation, pulse transit time, stroke volume, cardiac output, muscle contraction, heart rhythm, body temperature, skim temperature, triglycerides, skin conductivity, or any combination thereof.

12. The device of claim 9, wherein the one or more processors configured to identify one or more potential user conditions based on the one or more rates of change, the location of the mobile device and the user profile comprises one or more processor configured to:
identify a venue associated with the location of the mobile device;
determine whether the one or more rates of change correspond to one or more conditions related to the venue;
in response to the determination that the one or more rates of change are do not correspond to one or more conditions related to the venue, determine one or more potential user conditions based on the one or more rates of change and the user profile; and
associate the one or more potential user conditions with the venue.

13. The device of claim 9, wherein the one or more potential user conditions comprise diabetic reaction, choking, allergic reaction, asthma attack, hypertension, hypoglycemia, atrial fibrillation, tachycardia, a medication controlled medical condition, or any combination thereof.

14. The device of claim 9, wherein the one or more thresholds are based on a user-specific baseline, historical patterns of the one or more physiological parameters or any combination thereof.

15. The device of claim 9, wherein the one or more processors configured to provide one or more messages based on the identified one or more potential user conditions comprises one or more processors configured to:
request information based on the identified one or more potential user conditions;
receive one or more responses to the information request;
in response to the one or more responses indicating a user condition other than the one or more potential user conditions, store the one or more responses and the one or more rates of change.

16. The device of claim 15, wherein the one or more responses comprises a user consumed one or more beverages, consumed one or more foods, administered one or more medications, time of consumption, or any combination thereof.

17. A device for providing health assistance information, the device comprising:
means for determining one or more rates of change for one or more physiological parameters of a user;
means for determining whether the one or more rates of change meets one or more thresholds;
in response to the determination that the one or more rates of change meets one or more thresholds, means for identifying one or more potential user conditions based on the one or more rates of change, a location of the device and user profile; and
means for providing one or more messages based on the one or more potential user conditions.

18. The device of claim 17, wherein the device further comprises:
means for determining whether there is an emergency based on the identified one or more potential user conditions;
in response to the determination that there is an emergency, means for providing a notification of the emergency based on the identified one or more potential user conditions.

19. The device of claim 17, wherein the one or more physiological parameters comprises blood pressure, heart rate, glucose, respiratory rate, peripheral capillary oxygen saturation, pulse transit time, stroke volume, cardiac output, muscle contraction, heart rhythm, body temperature, skim temperature, triglycerides, skin conductivity, or any combination thereof.

20. The device of claim 17, wherein the means for identifying one or more potential user conditions based on the one or more rates of change, the location of the mobile device and the user profile comprises:
means for identifying a venue associated with the location of the mobile device;
means for determining whether the one or more rates of change correspond to one or more conditions related to the venue;
in response to the determination that the one or more rates of change are do not correspond to one or more conditions related to the venue, means for determining one or more potential user conditions based on the one or more rates of change and the user profile; and
means for associating the one or more potential user conditions with the venue.

21. The device of claim 17, wherein the one or more potential user conditions comprises diabetic reaction, choking, allergic reaction, asthma attack, hypertension, hypoglycemia, atrial fibrillation, tachycardia, a medication controlled medical condition, or any combination thereof.

22. The device of claim 17, wherein the one or more thresholds are based on a user-specific baseline, historical patterns of the one or more physiological parameters or any combination thereof.

23. The device of claim 17, wherein the means for providing one or more messages based on the identified one or more potential user conditions comprises:
means for requesting information based on the identified one or more potential user conditions;
means for receiving one or more responses to the information request;
in response to the one or more responses indicating a user condition other than the one or more potential user conditions, means for storing the one or more responses and the one or more rates of change.

24. The device of claim 13, wherein the one or more responses comprises a user consumed one or more beverages, consumed one or more foods, administered one or more medications, time of consumption, or any combination thereof.

25. A non-transitory computer-readable medium of a device for providing health assistance information comprising processor-executable program code configured to cause one or more processors to:
determine one or more rates of change for one or more physiological parameters of a user;
determine whether the one or more rates of change meets one or more thresholds;
in response to the determination that the one or more rates of change meets one or more thresholds, identify one or more potential user conditions based on the one or more rates of change, a location of the device and user profile; and
provide one or more messages based on the one or more potential user conditions.

26. The non-transitory computer-readable medium of claim 25, wherein the processor-executable program code is further configured to cause one or more processors to:
determine whether there is an emergency based on the identified one or more potential user conditions;
in response to the determination that there is an emergency, providing a notification of the emergency based on the identified one or more potential user conditions.

27. The non-transitory computer-readable medium of claim 25, wherein the one or more physiological parameters comprises blood pressure, heart rate, glucose, respiratory rate, peripheral capillary oxygen saturation, pulse transit time, stroke volume, cardiac output, muscle contraction, heart rhythm, body temperature, skim temperature, triglycerides, skin conductivity, or any combination thereof.

28. The non-transitory computer-readable medium of claim 25, wherein the processor-executable program code configured to cause one or more processors to identify one or more potential user conditions based on the one or more rates of change, the location of the mobile device and the user profile comprises processor-executable program code configured to cause one or more processors to:
identify a venue associated with the location of the mobile device;
determine whether the one or more rates of change correspond to one or more conditions related to the venue;
in response to the determination that the one or more rates of change are do not correspond to one or more conditions related to the venue, determine one or more potential user conditions based on the one or more rates of change and the user profile; and
associate the one or more potential user conditions with the venue.

29. The non-transitory computer-readable medium of claim 25, wherein the one or more potential user conditions comprises diabetic reaction, choking, allergic reaction, asthma attack, hypertension, hypoglycemia, atrial fibrillation, tachycardia, a medication controlled medical condition, or any combination thereof.

30. The non-transitory computer-readable medium of claim 25, wherein the one or more thresholds are based on a user-specific baseline, historical patterns of the one or more physiological parameters or any combination thereof.
